(19) United States
(12) Patent Application Publication
(10) Pub. No.: US 2024/0341371 A1
(43) Pub. Date: Oct. 17, 2024

(54) Garment for Support of the Gastrointestinal Tract and Posture

(71) Applicant: PGP Health Pty Ltd., Melbourne (AU)

(72) Inventor: Ardian Latifi, Melbourne (AU)

(73) Assignee: PGP Health Pty Ltd., Melbourne (AU)

(21) Appl. No.: 18/681,370

(22) PCT Filed: Aug. 2, 2022
(86) PCT No.: PCT/AU2022/050828

§ 371 Date: Feb. 5, 2024

(30) Foreign Application Priority Data
Aug. 5, 2021 (AU) 2021902422

(51) Int. Cl. A41B 9/06

(52) U.S. Cl. CPC A41B 9/06; A41D 2400/32; A41D 2400/38

(57) ABSTRACT

A garment for compressing and supporting the gastrointestinal (GI) tract and supporting posture, associated methods of use and treatment, including a method of promoting weight reduction, the method comprising using a medical garment that simultaneously compresses and lifts the GI tract.

### FIELD OF THE INVENTION

The present invention relates to a garment for compressing and supporting the gastrointestinal (GI) tract and support posture, and methods of use and treatment.

### BACKGROUND OF THE INVENTION

Gastrointestinal diseases (caused by pathogens, uncontrolled inflammation, physiological and morphological abnormalities, genetics, gut dysbiosis), and metabolic syndrome (such as obesity) have become prevalent around the world causing high morbidity and mortality rate.

Some of the factors contributing to gastrointestinal (GI) diseases are non-steroids anti-inflammatory drugs, genetic predisposition, unhealthy lifestyle, use of antibiotics, pathogens etc.

Most common GI diseases include irritable bowel disease, Crohn's disease, celiac, ulcerative colitis, bloating, acid reflux, constipation, abdominal pain, irregular bowel syndrome, fluid retention and a range of GI-related cancers.

Current treatment for GI diseases includes opioids, motility enhancers, anti-inflammatory agents, laxatives, anti-diarrheal, anti-emetics, change in diet and anti-cancer treatment.

Metabolic syndrome is associated with poor diet, age, ethnicity, unhealthy lifestyle, and, as a condition, correlates with increased risk for cardiovascular diseases, diabetes, stroke, hypertension, and other health diseases.

Approximately 60% of the world's population is affected by being overweight or obese and, 20% suffer from acid reflux, 30% from dysplasia, 12% from constipation, 0.3% from irritable bowel disease, 11.2% from irregular bowel syndrome, 26% from GI-related cancers, and 1.7 billion cases of childhood diarrhoeal every year.

The underlying causes of these diseases are often life threatening if not treated early on; at least 2.8 million people die each year as a result of complications related to being obese. Such serious complications include a risk of type 2 diabetes, cardiovascular medical conditions, hypertension, certain cancers (breast, colon, endometrial, and gastrointestinal tract), stroke and autoimmune diseases. Furthermore, approximately 1.6 million people died from diarrheal diseases in 2017 globally and there were 3.4 million related deaths worldwide.

Social and environmental factors contribute to obesity and other GI related diseases, including smoking, stress, alcohol consumption, unhealthy diets, and lack of physical activity. For example, it has been demonstrated that physical inactivity combined with poor sitting posture, i.e. at work, clearly causes rapid weight gain.

A distinguishing body characteristic of obese individuals is a pear-shaped type waistline as shown in FIG. 1. The larger the waistline the higher the risks of the serious complications mentioned above.

These serious complications arise from the accumulation of visceral fat stored in the lower end of the GI tract, abdominal area/midsection or belly. This fat is different to the fat that accumulates around other parts of the body such as arms, hips, and thighs. The visceral fat is an endocrine organ capable of synthesizing many types of hormones and chemicals including large amounts of toxic substances.

The accumulation of these toxic substances produced by the visceral fat can cause size alterations of the small and large intestine, loss of microbial flora as a result of antibiotic treatment, build-up of toxic waste and gases, micro inflammation, excess fluid and so on. The toxic substances can also block the blood vessels within the abdominal wall, or can accumulate outside the abdominal wall.

It is common knowledge that narrowing or blocking of the blood vessels leads to hypertension, cardiovascular disease and inflammation in many parts of the body. In particular, visceral fat compromises the immune system that produces cytokines such as interleukin-6 (IL-6), IL-1 and tumor necrosis factor (TNF). Finally, metabolic pathways disturbances are also associated with accumulation of visceral fat.

Furthermore, the visceral fat accumulated over longer periods is difficult to reduce due to its harmful consequences and physiological changes, as described above.

From the foregoing, it will be appreciated that there is a need to reduce body weight, enhance detoxification, reduce excess fluid and gases, suppress the appetite, reduce bloating and inflammation, and gradually allow the body to heal itself from the ongoing damages caused over time.

Compression therapy has long been used as an effective means to treat a number of medical conditions via increasing blood flow activity in legs and improving overall blood flow to the heart, decreasing swelling and reducing local inflammation. Similarly, compression postures used in yoga practices have been shown to improve digestion through compressing the stomach and lower abdomen.

The present invention sets out to address some of the above issues, and to remedy at least in part the shortcomings of the prior art, by providing a wearable product that assists in improving the GI tract performance.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention has been born from the inventor's realisation that both the compression and lifting of at least the lower GI tract improves functioning. In addition, that it would be beneficial to improve the posture of these people by supporting their upper and lower back, as poor posture is often caused by heavy weight in and around the lower GI tract.

According to a first aspect, there is provided a garment for lifting and compressing to enhance the function of the gastrointestinal (GI) tract and support posture, said garment comprising:
a front portion configured to receive the ventral part of a wearer, wherein the front portion comprises at least a central front section comprising a first material such that, when worn by the wearer, the garment lifts and compresses at least the lower GI tract at the same time; and
a rear portion configured to receive the dorsal part of the wearer, such that, when worn by the wearer, the rear portion is positioned to compress and/or support the dorsal part of the wearer further and supports the lifting of the lower GI tract by the front portion.

In an embodiment, there may be provided at least one panel comprising a second material, the panel(s) layered with at least the rear portion, the panel(s) extending such that, when worn by the wearer, the panel(s) is/are positioned to compress and/or support at least the dorsal part of the wearer.

The front portion may comprise a central front section and at least two lateral front sections.

In an embodiment, there may be provided a central elastic spine incorporated into the rear portion, such that, when worn by the wearer, the central elastic spine is positioned to provide spinal support. The rear portion may comprise at least two rear sections. The at least two rear sections may be provided in the first material.

Preferably, the second material has relative stiffness greater than the first material. The first material may be a 100% synthetic fabric, preferably comprised of nylon and elastane, such as Spandex. The first material may be a liquid titanium fabric. The first material preferably has a 4-way stretch.

The second material preferably also has a 4-way stretch. Advantageously, the second material is a power mesh fabric. The power mesh fabric may be made from polyester, nylon and elastomeric fabric.

The central elastic spine is made from a third material that preferably has a relative stiffness greater than the first material, and ideally also the second material. The central elastic spine extends the full height of the garment running centrally along the rear portion. In one embodiment, the at least two rear sections are lateral sections joined to either side of the central elastic spine. Ideally, the third material has a 2-way stretch that runs vertically to provide lateral support to the centre of the garment and the wearer's back, whilst allowing for the wearer to bend forward when wearing the garment.

The garment preferably forms a vest structure that is pulled down over the body of the wearer, with the central front section providing the required stretch to enable the vest to be pulled over the stomach region easier compared to the rest of the panels.

The at least one panel may be layered on the outside of the front and/or rear portions, but more preferably are layered inside. They may be stitched through the two layers to avoid movement of the layers with respect to each other. The at least one panel may be two panels extending laterally from either side of the spine to at least the edges of the rear portions. The panels may extend from the spine around to the edges of the central front section.

There may be provided two sets of panels, one above the other to form an upper set of panels and a lower set of panels. The upper set of panels may extend from the spine in a downward angle towards the edges of the rear portions. The upper set of panels may be positioned to line up underneath the wearer's shoulder blades.

The lower set of panels may lie at or adjacent the lower hem of the garment. In one embodiment, the lower set of panels may begin above the hem of the garment at the spine and curve downwardly towards the hem at the front of the garment. Advantageously, the lower set of panels are positioned to align with the lower gastrointestinal tract (lower GI) and provide compression and an element of lifting in that region.

There may be provided an additional compression element that may take the form of a belt extending across the central front section. In an embodiment, the belt provides an extension to join the ends of the lower set of panels and complete the compression loop around the lower GI region. The belt may be made of any suitable fabric that is ideally of greater stiffness to the second material, and may be made by layering multiple layers of the second material to create the belt. One end of the belt is preferable stitched to an edge of the front central region and the other end extends over the opposing lateral front section and is fastenable thereto. The fastener may be, for example, hook and loop fastener, which allows the degree of compression to be adjusted by the wearer.

The shape of the vest may be such that the armholes are provided such that they are further cut in at the back than the front, which, in combination with the rest of the garment features, results in the wearer's shoulders being pulled back.

According to a second aspect, there is provided a method for compressing and supporting the gastrointestinal (GI) tract of a wearer, said method comprising:
lifting and compressing the GI tract of the wearer by having them wear a garment according to the first aspect of the invention. The present invention may also extend to a method of treating a person to improve GI health, by wearing the garment according the first aspect of the invention.

According to a third aspect of the invention there is provided a method of promoting weight reduction, the method comprising using a medical garment that simultaneously compresses and lifts the GI tract.

According to a fourth aspect of the invention there is provided a method of using a garment to improve the GI function of a wearer, the method including:
providing a garment that simultaneously compresses and lifts the GI tract of the wearer;
having the wearer wear the garment for at least 8 hours per day on average for at least 5 consecutive days.

Ideally, the wearer increases the duration of time from 8 hours per day over time to up to 24 hours.

According to a fifth aspect of the invention there is provided a garment according to the first aspect used as a device in the treatment of at least one of the following physiological and morphological abnormalities: metabolic disorders such as obesity; water retention; irregular bowel movements; inflammatory diseases such as irritable bowel syndrome, ulcerative colitis, celiac, Crohn's disease); bloating; dyspepsia; gastrointestinal reflux disease; low energy levels; large appetite; dysbiosis; hernia; poor posture related issues such as back pain, hand numbness, spinal dysfunction, joint degeneration, headaches, poor balance, breathing difficulties, rounded shoulders, potbelly; urinary incontinence; constipation; heartburn; or slow digestion.

According to sixth aspect of the invention there is provided a method for the treatment of obesity, comprising providing the patient with at least a first garment according to the first aspect, placing the wearer in the garment, instructing them to wear the garment for at least 8 hours per day; adjusting the compression level of the garment they are wearing over time to increase the compression of the GI tract.

According to the sixth aspect, the garment used preferably includes an adjustable belt across the wearer's midsection. Alternatively, the wearer may be provided with a number of garments of different compression ratings and sizes.

According to seventh aspect, there is provided a method for the treatment of at least one of the following physiological and morphological abnormalities: metabolic disorders such as obesity; water retention; irregular bowel movements; inflammatory diseases such as irritable bowel syndrome, ulcerative colitis, celiac, Crohn's disease); bloating; dyspepsia; gastrointestinal reflux disease; low energy levels; large appetite; dysbiosis; hernia; poor posture related issues such as back pain, hand numbness, spinal dysfunction, joint degeneration, headaches, poor balance, breathing difficulties, rounded shoulders, potbelly; urinary incontinence; constipation; heartburn; or slow digestion; the method including:
measuring the wearer's torso area;
choosing a garment according to the first aspect with the correct fit for the wearer and with the correct compression and/or lift and/or back support required;
instructing the wearer to wear the garment for an initial period of time;
assessing progress after the initial period of time;
   assessing whether a garment with greater compression is required for a second period of time.

### BRIEF DESCRIPTION OF THE DRAWINGS

Further aspects of the present invention and further embodiments of the aspects described in the preceding paragraphs will become apparent from the following description, given by way of example and with reference to the accompanying drawings.

FIG. 1 shows a representation of obese and overweight people as defined using the body mass index;

FIG. 2 shows a representation of: A, a normal functioning GI tract versus B, an overweight GI tract;

FIG. 3 is a front view of a garment according to a first embodiment of the invention;

FIG. 4 is a rear view of the garment of FIG. 3;

FIG. 5 is a front view of a garment according to a second embodiment of the invention, showing the internal layered panels for ease of illustration;

FIG. 6 is a rear view of the garment of FIG. 4;

FIG. 7 is a front view of a garment according to a third embodiment of the invention;

FIG. 8 is a rear view of the garment of FIG. 7;

FIG. 9 is a front view of a garment according to a fourth embodiment of the invention;

FIG. 10 is a rear view of the garment of FIG. 9;

FIG. 11 is a front view of a garment according to a fifth embodiment of the invention;

FIG. 12 is a rear view of the garment of FIG. 11;

FIG. 13 is a front view of a garment according to a sixth embodiment of the invention;

FIG. 14 is a rear view of the garment of FIG. 13;

FIG. 15 is a front view of a garment according to a seventh embodiment of the invention;

FIG. 16 is a rear view of the garment of FIG. 15;

FIG. 17 shows a representation of: C an improved functioning GI tract after the user has worn the garment of the present invention; and

FIGS. 18a and 18b are tables showing different sizes of the garment.

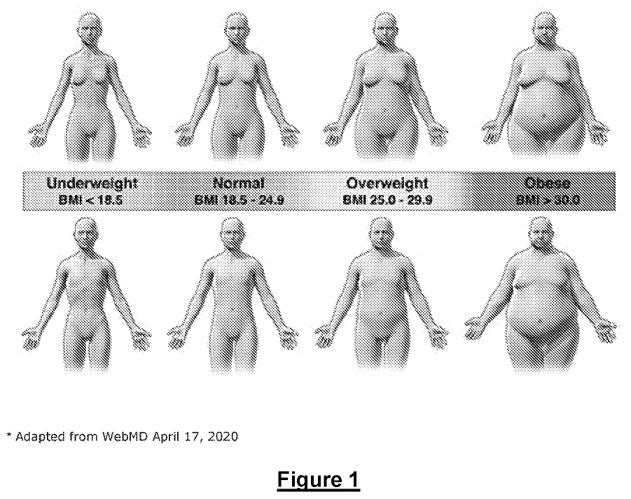

### DETAILED DESCRIPTION OF THE EMBODIMENTS

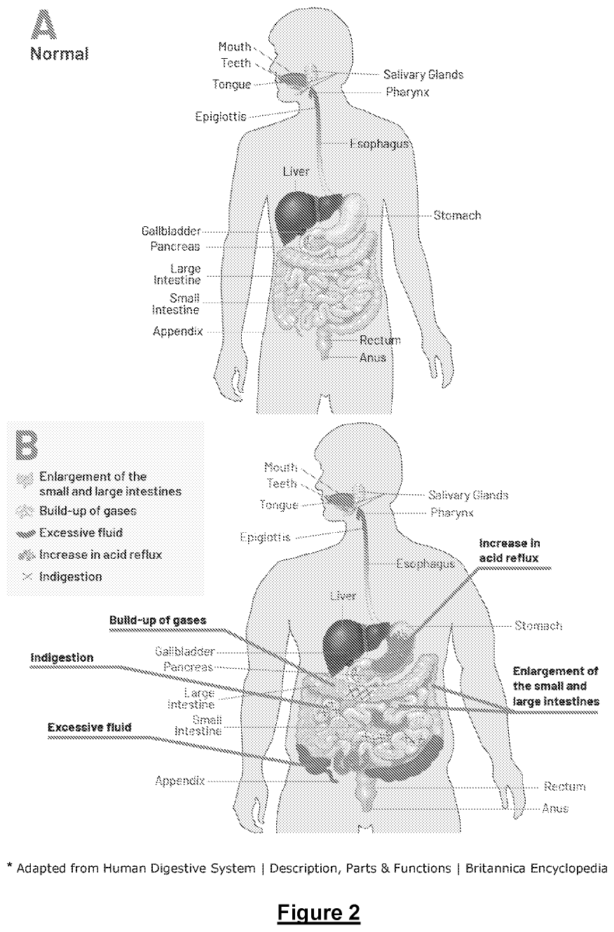

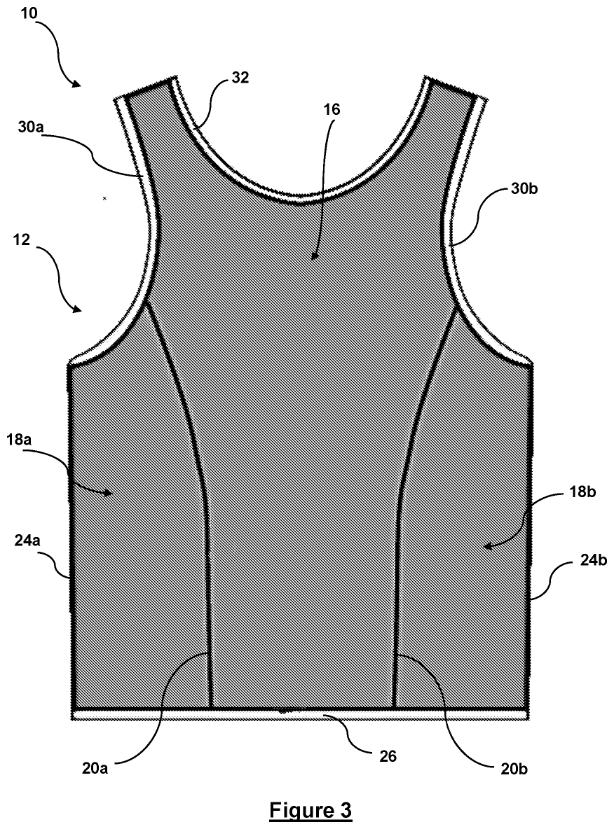

An embodiment of a garment 10 of the present invention is shown in FIG. 3. The garment 10 is a medical vest with a tube like structure to be worn by obese, overweight or others in need, to enhance detoxification, increase their metabolism/energy levels, regulate bowel movements, and reduce bloating and inflammation. Generally, as weight is gained, the lower GI tract, small and large intestine in particular, become physically extended due to build-up of toxins, gases, and excess fluid, causing pain and other physical abnormalities. This is illustrated in FIG. 2, which shows a representation of the changes in the GI tract between a normal functioning GI tract in picture A versus an overweight or obese GI tract in picture B. In turn, these changes in the GI tract slow down and repress the function of the GI tract causing a number of known diseases related to weight gain. The present inventors therefore aim to lift and compress the GI tract to allow for the physiological changes to reverse back to its normal state as a function of time and thus improving its functioning.

The inventors have realised that compressing the entire GI tract further supports its function by suppressing the appetite of the wearer, helping the body to eliminate any excess fluid/gases, supporting muscle contractions, and reduces inflammation and bloating. Furthermore, lifting the GI allows the large and small intestine, and pelvic organs to function better in a similar fashion to a normal physiological condition without any hindrance from the heavy weight that is accumulated in this region.

The present invention arises from a realisation by the inventors regarding the beneficial effect of the compression and lifting properties to improve the health and performance of the GI tract. The inventors have observed that the effect of waistline shapers with a compression property only, caused an increase in acid reflux, a painful feeling around the region and difficulty in breathing. Hence, the present invention provides an alternative and novel approach to improve GI tract and posture by gradually reversing the physiological changes that may have occurred over a long period of time due to poor posture, lack of exercise, poor diet, stress, alcohol, underlying genetics, and other influential factors, in a natural way whilst avoiding the above-mentioned complications.

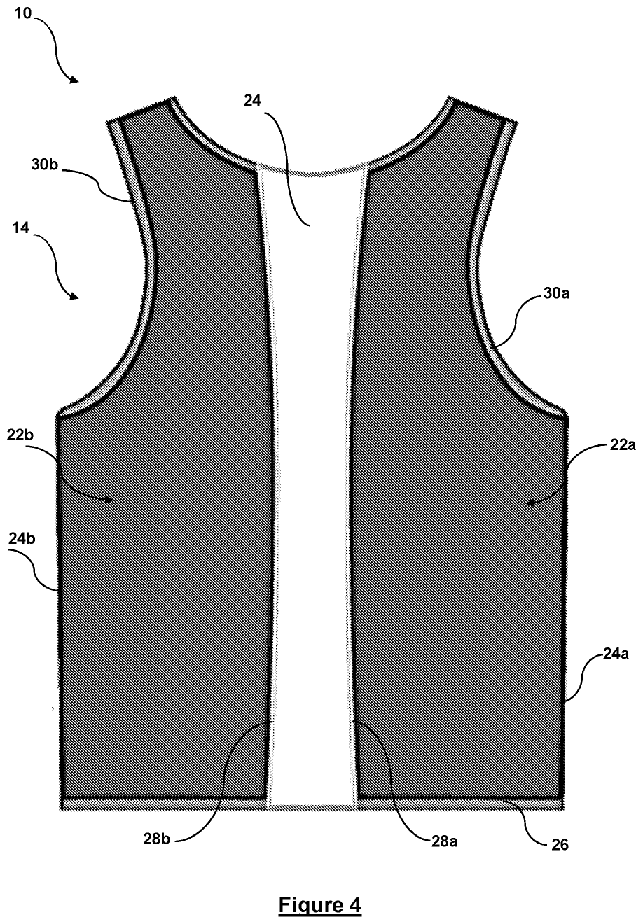

The garment 10 comprises a front portion 12 (shown in FIG. 3) and a rear portion 14 (shown in FIG. 4). The front portion 12 covers the ventral part of the wearer while the rear portion 14 covers the dorsal part of the wearer. In the embodiment illustrated in FIG. 3, the front portion 12 is constructed from a central front section 16 positioned along the centre of the front portion 12 with two lateral front sections 18a, 18b sewn laterally to either side of the central front section 16. The edges of the central front section 16 create seams 20a, 20b.

The rear portion 14 shown in FIG. 4 comprises two rear sections 22a, 22b, which extend laterally away from a central elastic spine 24. The spine 24 is represented in a contrasting colour in the drawings to represent a different material to the other portions, which will be described further below.

The central front section 16 of the front portion 10 and the rear sections 22a, 22b of the rear portion 14 are created as a continuous piece of fabric extending over the shoulders of the wearer, so there are no shoulder seams. The two lateral front sections 18a, 18b and the rear sections 22a, 22b are connected at side seams 24a, 24b, respectively.

It will be understood that in other embodiments of the present invention the two lateral front sections 18a, 18b and the rear sections 22a, 22b can extend as a continuous piece of fabric without side seams.

The garment may be made primarily of a first material such as a 100% synthetic fabric, preferably comprised of nylon and elastane, such as Spandex. This is illustrated by the grey sections in FIGS. 3 through 6. Furthermore, the first material may be a liquid titanium fabric. In the present embodiment the fabric used to create the vest consist of 75% liquid titanium and 25% Spandex.

The properties of liquid titanium include: high UV rating of 50+, soft and comfortable feel, excellent stretch and shape retention, high retaining capacity, excellent 4-way stretch, water repellent, and it is a quick-dry fabric.

It is known that liquid titanium fabrics increase blood flow with and without compression. It radiates heat and increases body temperature on the subcutaneous layer of the skin, boosting the immune system, provides pain relief and detoxification, increases metabolism and energy levels, has antibacterial/antimicrobial properties, and deodorization. As such, this fabric is widely used in many products such as headbands, sporting apparel, wrist bands and other joint bands.

The front and rear portions 12, 14 of the garment 10 are configured to create lift and compression throughout the GI tract and in particular in the abdominal region about the lower GI tract. The 4-way stretch property of the liquid titanium fabric around the central front section 16 works by a vertical pulling approach from a lower hem 26. The material provides the required stretch to pull the vest over the entire large and small intestine, and, as a result, this has an impact on the stomach of the wearer. Once the vest is pulled over the lower GI tract, the compressive nature of the first fabric combined with the garment being a vest that extends over the shoulders of the wearer, results in lifting and compressing of the entire GI tract region.

In order for the vest to be effective, in most cases, at least 4 cm of the vest needs to stretch (be pull) down over the wearer's stomach, which in turn will cause sufficient lift on the GI tract.

At the rear portion 14, the 4-ways stretch of the liquid titanium pulls the upper body backwards and the lower part of the back inward, providing back support. The central elastic spine 24 extends the full height of the garment 10 running centrally along the rear portion 14. In the present embodiment, the central elastic band joins the two rear sections 22a 22b at seams 28. The central elastic spine 24 is made of a material that preferably has a relative stiffness greater than the first material. Ideally, this fabric has a 2-way stretch that runs vertically along the spine to provide lateral spinal support to the centre of the garment and the wearer's back, whilst allowing for the wearer to bend forward whilst wearing the garment.

Additional postural support is provided with armholes 30a, 30b further cut in at the rear than the front to pull back the shoulders of the wearer to correct inappropriate posture. This in turn enables the wearer to maintain an overall adequate posture and brings back the physiological changes to an original shape and form, in a gradual and safe manner, improving the overall health of the wearer. An improved posture assists in improving GI tract function.

The garment construction includes additional finishes to further complement the aesthetic and functional features, such as fold over elastic extending along the neckline 32, armholes 30a, 30b and the lower hem 26 of the garment, respectively. The lower hem 26 may include a silicone strip along the inside surface to gently grip the wearer's skin preventing the garment from lifting during use.

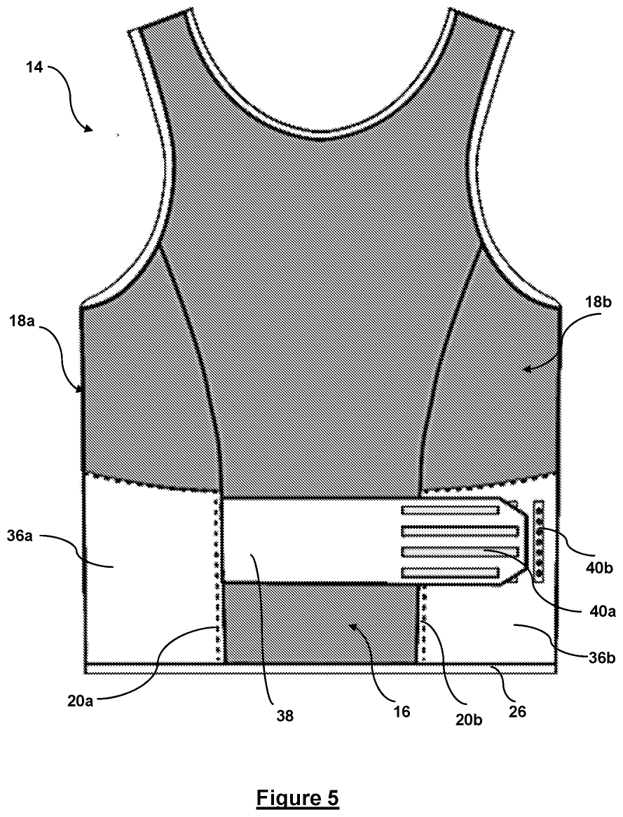

Additional beneficial compression in the garment can be provided by panels specifically shaped and positioned to increase the effectiveness of the garment. These panels are represented in the embodiment shown in FIGS. 5 and 6. Whilst it will be appreciated that the panels may be positioned either internally or externally to the first fabric layers of the front and rear portions, they are preferably positioned on the inside surface. However, for ease of representation, in FIGS. 5 and 6, the panels are visible in the drawings. Stitching extends through the two layers of fabric to avoid movement of the layers with respect to each other.

Whilst only one set of panels may be provided, the present embodiment includes two sets, an upper set and a lower set. The panels are preferably made of a second material or double layers of the second material. The second material may be a power mesh fabric with a 4-way stretch, and ideally has a relative stiffness that is greater than the first material. The power mesh fabric may be made from polyester and nylon. It is known that power mesh is a lightweight and breathable material that is suitable for active wear as it allows moisture to escape and keeps your body cool. These additional panels of stiffer material are intended to provide additional compression and support to the garment at key locations.

Turning to FIG. 6, which shows the rear of the garment, it can be seen that the elastic spine 24 runs centrally from the neckline 32 to the hem 26. The spine 24 is slightly wider at the neckline and gradually curves inwardly along the centre of the wearer's spine.

An upper set of panels 34a, 34b extend from the side seams 28a, 28b of the spine 24 in a downward angle towards the edges of the rear portions to side seams 24a, 24b. The upper set of panels may be positioned to line up underneath the wearer's shoulder blades. This upper set of panels of relatively stiffer material assists in improving the wearer's posture and also transfers some of the weight of the wearer's lower GI tract and stomach to the upper shoulder section.

A set of lower panels 36a, 36b lie adjacent the lower hem 26 of the garment 10, extending laterally from side seams 28a, 28b of the spine 24. The panels 36a, 36b begin slightly above the lower hem 26 of the garment 10 to sit anatomically above the outward curve of a wearer's buttocks. The panels 36a, 36b then curve downwardly towards the lower hem 18 as they wrap around the wearer's torso. As shown in FIG. 5, the panels 36a, 36b extend around to the front portion 14 and cease at the front seams 20a, 20b of the central front section 16.

Due to the above mentioned features, the present invention has advantageously aligned the panels 26a, 26b with the lower GI tract to provide a further element of compression, whilst providing a further element of lifting and support to the muscles, ligaments, joints and discs. These anatomical structures may have been injured due to inappropriate posture whilst sitting in front a computer, physical injuries, hyper immunity and so on; thus, gradually allowing the body to heal itself.

It will be noted that in this illustrated embodiment, the panels 36a, 36b do not extend across the central front section 16 and the wearer's stomach. This provides the required stretch and flexibility for the wearer to get into the garment and stretch the vest over the stomach region. To further enhance the compression and lifting of the stomach region, an adjustable belt 38 extends across the central front section 16. The adjustable belt 38 extends around the lower GI region from seam 20a over the front section 16, and past opposing seam 20b to fasten to the opposite lateral front section 18b. In the present embodiment the fastener is in the form of male Velcro 40a positioned on the internal side of the adjustable belt 38. The male Velcro 40a fastens to a female Velcro 40b on the outside of the lateral front section 18b, as shown in FIG. 5. It is understood that the fastener may be any suitable fastener that allows the degree of compression to be adjusted by the wearer.

In the present embodiment the adjustable belt 38 is made of four layers of power mesh to provide an additional compression element around the lower GI region. It is understood that the belt may also be made of any suitable fabric that is ideally of greater stiffness than all other materials used, and may be made by layering multiple layers material to create the belt.

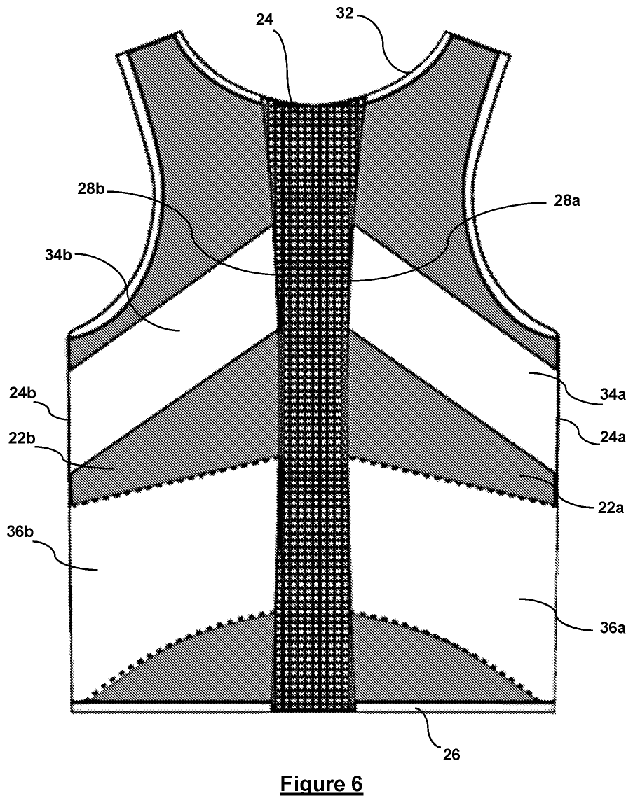

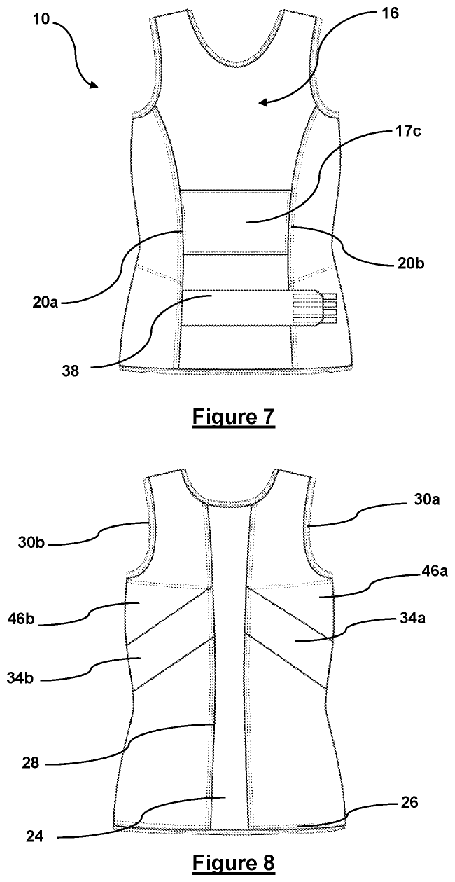

FIGS. 7 and 8 show a front and rear view of a different embodiment of the present invention. The features of garment 10 in FIGS. 7 and 8 that have already been disclosed in the embodiments presented in FIGS. 3 to 6 will not be further discussed.

In FIG. 7 adds additional compression to the upper GI region with an underlay panel 17c extending across a midway point of the central front section 16 from seam side 20a to seam side 20b. This additional compression to the upper GI region promotes the suppression of the wearer's appetite, which assists in the wearer's weight loss journey.

In FIG. 8 a set of lower panels 46a, 46b lie adjacent the lower hem 26 of the garment 10, extending laterally from side seams 28 of the spine 24 and extend longitudinally up to the bottom of armholes 30a, 30b in order to provide compression to the upper part of the Gl. The lower panels 46a, 46b are optimally constructed as internal panels of power mesh, whilst the out layer of the garment is made from liquid titanium fabric. An upper set of panels 34a, 34b are also included in this embodiment, as is a power mesh belt 38.

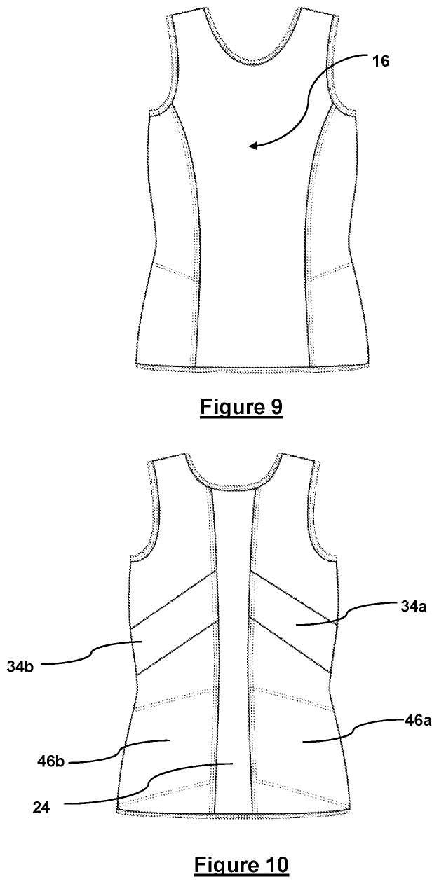

The embodiment of the garment 10 shown in FIGS. 9 and 10 could be used as a sport or lighter version of the embodiment presented in FIGS. 5 through 8, similar to the version shown in FIGS. 3 and 4, as it has lighter compression than other versions. The lifting and back support is provided by the set of lower panels 46a, 46b and upper set of panels 34a, 34b, which have been described above.

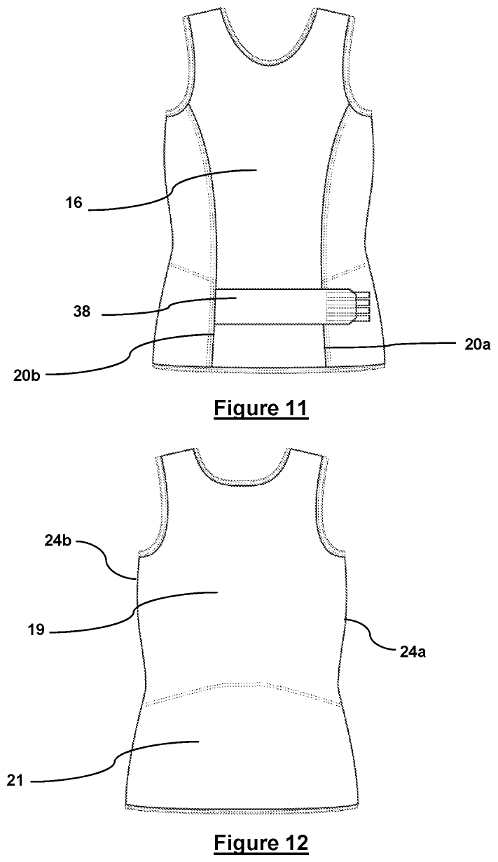

The embodiment of the garment 10 shown in FIGS. 11 and 12 is designed for individuals who are not severely overweight. These individuals generally don't need additional spine or back support. In particular, the rear part of garment comprises one rear section 19 which extend laterally from seam 24a to seam 24b. A lower internal panel 21 lies adjacent the lower hem 26 of the garment 10, extending laterally from seam 24a to seam 24b. The panel 21 begin on the lower hem 26 of the garment 10 to sit anatomically on the outward curve of a wearer's buttocks. As shown in FIG. 12, the panel 21 extends around to the front portion 14 and cease at the front seams 20a, 20b of the central front section 16. This embodiment may include a belt 38. It may additionally include an upper GI panel, similar to that shown the embodiment of FIG. 7.

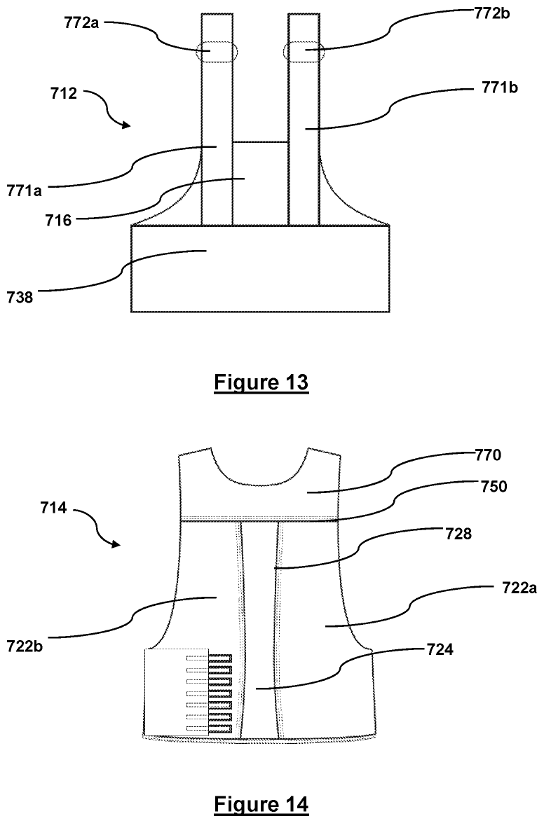

FIGS. 13 and 14 show a further embodiment whereby the garment 10 comprises a front portion 712 and a rear portion 714. The front portion 712 covers the ventral part of the wearer while the rear portion 714 covers the dorsal part of the wearer. The front portion 712 is constructed from a central front section 716 and a lower panel 738 positioned across the stomach of the wearer. Buckled straps 771a, 771b extend vertically along the central front section 716, joining to the top of front section 716. The buckled straps 771a, 771b comprise buckle adjustment 772a, 772b to provide adjustable lifting of the wearer's stomach region.

The central front section 716 and buckled straps 771a, 771b may be made primarily of a static material such as cotton drill. The lower panel 738 being made from an elastic material to provide compression across the lower GI tract.

The rear portion 714 comprises rear sections 770, 722a, 722b (FIG. 14). Rear section 770 covers the upper dorsal part of the wearer, extending from side seam 724a to side seam 724b. Rear section 770 may be made primarily of a static material such as cotton drill.

The rear section 770 of the rear portion 714 and the buckled straps 771a, 771b of the front portion 712 are created as a continuous piece of fabric extending over the shoulders of the wearer, so there are no shoulder seams.

Lateral rear sections 722a, 722b extend laterally away from a central elastic spine 724 (FIG. 14). The two lateral rear sections 722a, 722b are connected to the central elastic spine 724 at seams 728. The two later rear sections 722a, 722b and the central elastic spine 724 join the rear section 770 at seam 750.

The central elastic spine 724 is made of a material that has a 1-way stretch that runs vertically along the spine to provide lateral spinal support to the centre of the garment and the wearer's back, whilst allowing for the wearer to bend forward whilst wearing the garment. Rear sections 722a, 722b may be made primarily of a static material such as cotton drill.

To further enhance the compression and lifting of the stomach region, the belt 738 is adjustable and extends around the lower GI region to fasten the garment to the lateral rear section 722b. The belt may be made of any suitable elastic fabric.

In the present embodiment the fastener is in the form of Velcro however it is understood that the fastener may be any suitable fastener that allows the degree of compression to be adjusted by the wearer, such as hook and eye closures.

It will be understood that a further embodiment of the present invention may differ from the embodiment in FIGS. 13 and 14 by having a rear portion 714 not equipped with a central elastic spine and only a single rear panel, in a static material, positioned between side seams.

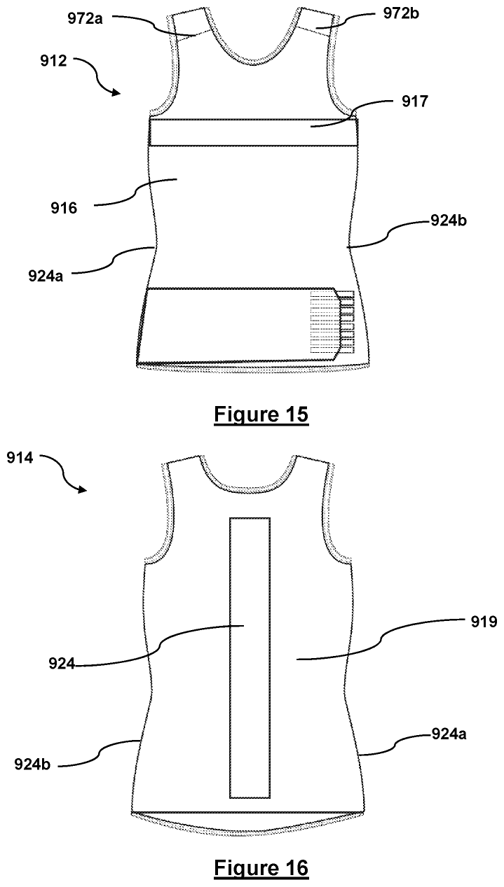

FIGS. 15 and 16 show a front and rear view of another embodiment of the present invention. In this embodiment, the garment 10 comprises a front portion 912 (FIG. 15) and a rear portion 914 (FIG. 16). The front portion 912 covers the ventral part of the wearer while the rear portion 914 covers the dorsal part of the wearer. The front portion 912 is constructed from a front section 916 positioned along front portion 912.

An upper panel 917 extends longitudinally in the upper part of the central front section 916 from seam side 924a to seam side 924b. It is understood that the upper panel 917 provides additional lifting and compression across the upper torso of the wearer. The front section 916 and the upper panel 917 may be made primarily of a lightweight material such as coolmesh.

The rear portion 914 of the embodiment in FIG. 16 comprises a panel 919 as a continuous piece of fabric that extends from seam side 924a to seam side 924. Panel 919 further extends over the shoulders of the wearer wherein Velcro fasteners 972a, 972b fasten on the front section 916 allowing the degree of lifting to be adjusted by the wearer. Panel 919 may be made primarily of a stretch static fabric.

A central elastic spine 924 in FIG. 16 is joined longitudinally in the rear portion 914. The central elastic spine 924 is made of a material that has a 1-way stretch that runs vertically along the spine to provide lateral spinal support to the centre of the garment and the wearer's back, whilst allowing for the wearer to bend forward whilst wearing the garment.

It is understood that further embodiments of the present invention can comprise different combination of the front and rear portion of the embodiments disclosed in FIGS. 3 through 16.

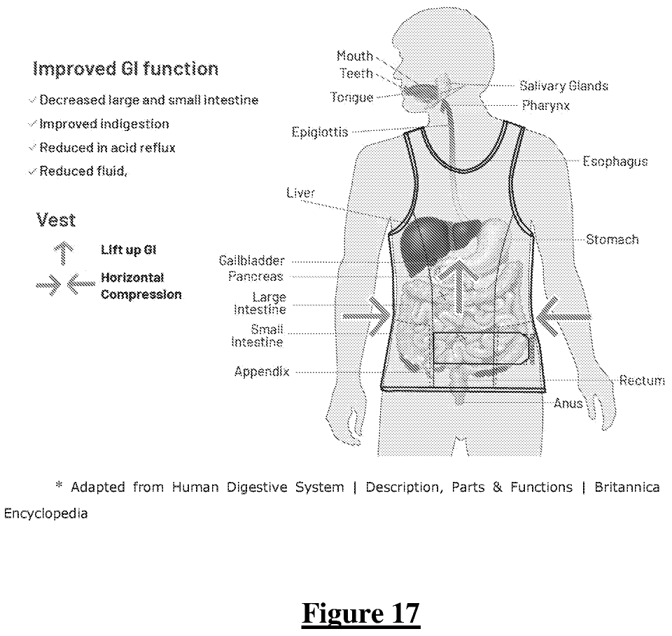

FIG. 17 shows a representation of the vest according to the present invention on a wearer showing the GI tract. The arrows indicate the horizontal, radial compression around the lower region of the garment and the lifting of the GI tract vertically to improve GI function.

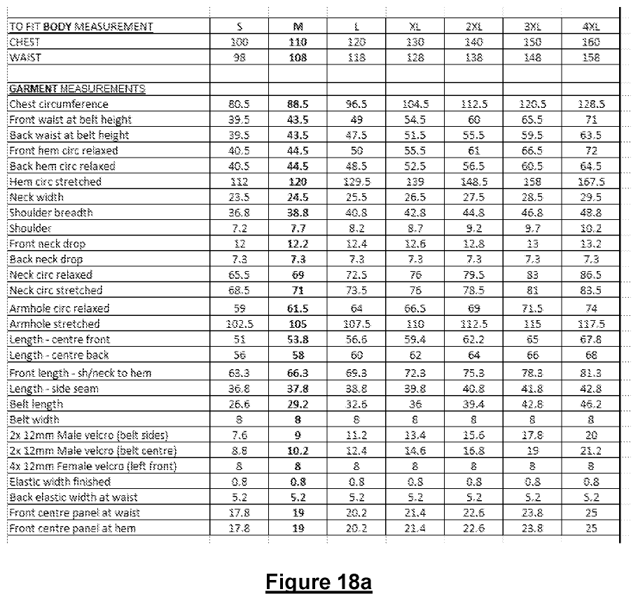

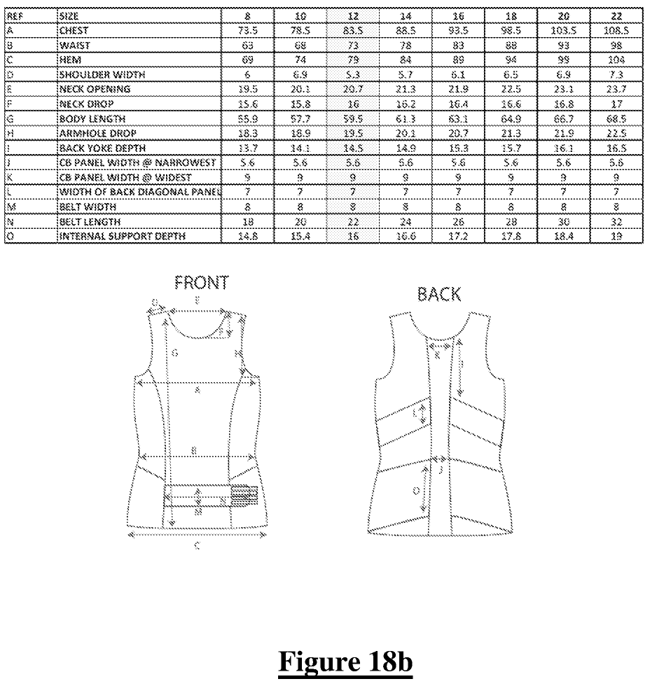

FIG. 18a shows a table comprising different sizes of the garment 10 to accommodate male wearers of different body sizes and shapes. FIG. 18b shows another table comprising different sizes to accommodate female wearers of different body sizes and shapes. As a user reduces in size due to weight reduction they may need to move down a size in garment. Users may commence with a garment having minimal compression whilst their body gets used to wearing the garment. They may begin with a garment that incorporates the adjustable belt, such that over time they can increase the compression and lifting. When reducing in size, it may be recommended that they also move to a garment having a greater level of compression, including one with specific upper GI compression to suppress the appetite.

The present invention may also extend to a method of treating a person to improve the GI health by wearing the garment. In a small cohort of less than 10 individuals wearing the vest from 4 hours a day, and progressing to 8 hours a day, then 12 hours a day, the inventors have observed, after 1 week, an improvement in bowel movement, reduction in waistline by 2.5 cm and overall weight of 1.5-2 Kg, reduction of acid reflux and improved posture. These numbers are increased over a 4 week period, depending on the original size of the wearer.

The present invention provides a wearable garment that can be worn by overweight, obese and those in need of improving the function of GI tract going about their everyday lives, underneath their clothing, that is providing medical treatment or enhance their physical performance as they wear it. The garment improves a wearer's posture, which itself has an impact on the functioning of the GI system, lifts the lower GI tract to a better functioning position, and provides compression to further improve its function and reduce unhealthy fluid.

### Case Study Examples

The following case study examples highlight the beneficial effect of the compression and lifting properties of the present invention to improve the health and performance of the GI tract. Generally, the overall improvements noted by wearer's included: reduced back pain, improved posture, reduction in inflammation, reduced bloating, reduced swelling in joints, weight loss, improved energy levels, regulation in bowel movements, reduced acid reflux, among others.

Case study 1: A female in their 30s, working as a health evaluator, wore the garment for 3 weeks, generally 24 hours a day. The woman observed health improvements after the first 3-4 days. Prior wearing the garment, the woman had severe back pain (rated 9/10), accompanied by bloating and water retention. After 3 weeks, the woman lost 2-3 kg. The garment also helped reducing bloating, fluids and gases. The woman reported significantly reduced pain the lower part of the back (rated 5/10).

Case study 2: A male in their 30s, working in the film and television industry, wore the garment for 5-6 months, generally 24 hours a day. The man observed health improvements after the first 7-8 days. Prior to wearing the garment, the man had bad posture and severe back pain (rated 9/10). The man also suffered from bloating and gastroesophageal acid reflux. After 5-6 weeks, the garment helped reducing bloating and significantly reduced the occurrence of acid reflux. Back pain reduced (rated 7/10).

Case study 3: A male in their 50s, working as accountant, wore the garment for 8 months, generally 24 hours a day. The man observed health improvements after the first 5-6 days. Prior to wearing the garment, the man had bad posture. The man was also overweight and suffered from bloating. As a beneficial effect of the garment, the man had improved the posture and lost 7 kg.

Case study 4: A male in their 70's, a pensioner, wore the garment for 16 months, not continuously. The man observed health improvements after the first 7-10 days. Prior wearing the garment, the man had bad posture. The man also suffered from obesity, bloating and was on medication for gastroesophageal acid reflux. As a beneficial effect of the garment, the man lost 8 kg and 6 cm around the body circumference. Furthermore, the man was less bloated, reduced his appetite and the medication for gastroesophageal acid reflux were no longer required.

Case study 5: A female in the 50s, a business owner, wore the garment for 1 month on and off. Prior to wearing they had major back problems due to extra weight and suffered from stool leaks due to Irritable bowel syndrome. After wearing the garment for 5-6 days they no longer experienced stool leaks and noticed an overall improvement of the GI function.

It will be understood that the invention disclosed and defined in this specification extends to all alternative combinations of two or more of the individual features mentioned or evident from the text or drawings. All of these different combinations constitute various alternative aspects of the invention.

1. A garment for lifting and compressing to enhance the function of the gastrointestinal (GI) tract and support posture, said garment comprising:
a front portion configured to receive the ventral part of a wearer, wherein the front portion comprises at least a central front section comprising a first material such that, when worn by the wearer, the garment lifts and compresses at least the lower GI tract at the same time; and
a rear portion configured to receive the dorsal part of the wearer, such that, when worn by the wearer, the rear portion is positioned to compress and/or support the dorsal part of the wearer further and supports the lifting of the lower GI tract by the front portion.

2. The garment according to claim 1, further comprising at least one panel comprising a second material, the panel(s) layered with at least the rear portion, the panel(s) extending such that, when worn by the wearer, the panel(s) is/are positioned to compress and/or support at least the dorsal part of the wearer.

3. The garment according to claim 1, wherein the front portion comprises a central front section and at least two lateral front sections.

4. The garment according to any claim 1, further including a central elastic spine incorporated into the rear portion, such that, when worn by the wearer, the central elastic spine is positioned to provide spinal support.

5. The garment according to claim 4, wherein the rear portion comprises at least two rear sections provided in the first material.

6. (canceled).

7. The garment according to claim 2, wherein the second material has relative stiffness greater than the first material.

8. The garment according to claim 1, wherein the first material is a 100% synthetic fabric, preferably comprised of nylon and elastane.

9. The garment according to claim 7, wherein the second material is a power mesh fabric.

10. The garment according to claim 4, wherein the central elastic spine is made from a third material that has a relative stiffness greater than the first material.

11. The garment according to claim 10, wherein the third material has a 2-way stretch that runs vertically to provide lateral support to the centre of the garment and the wearer's back, whilst allowing for the wearer to bend forward when wearing the garment.

12. The garment according to claim 4, wherein the central elastic spine extends the full height of the garment running centrally along the rear portion.

13. The garment according to claim 5, wherein the at least two rear sections are lateral sections joined to either side of the central elastic spine.

14. The garment according to claim 1, wherein the garment forms a vest structure that is pulled down over the body of the wearer, with the central front section providing the required stretch to enable the vest to be pulled over the stomach region easier compared to the rest of the panels.

15. (canceled).

16. The garment according to claim 2, wherein there is provided two sets of panels, one above the other to form an upper set of panels and a lower set of panels.

17.-21. (canceled).

22. A method for compressing and supporting the gastrointestinal (GI) tract of a wearer, said method comprising:
lifting and compressing the GI tract of the wearer by having them wear a garment according to claim 1.

23. A method of promoting weight reduction, the method comprising using a medical garment that simultaneously compresses and lifts the GI tract.

24. A method of using a garment to improve the GI function of a wearer, the method including:
providing a garment that simultaneously compresses and lifts the GI tract of the wearer;
having the wearer wear the garment for at least 8 hours per day on average for at least 5 consecutive days.

25. The garment according to claim 1 when used as a device in the treatment of at least one of the following physiological and morphological abnormalities: metabolic disorders such as obesity; water retention; irregular bowel movements; inflammatory diseases such as irritable bowel syndrome, ulcerative colitis, celiac, Crohn's disease); bloating; dyspepsia; gastrointestinal reflux disease; low energy levels; large appetite; dysbiosis; hernia; poor posture related issues such as back pain, hand numbness, spinal dysfunction, joint degeneration, headaches, poor balance, breathing difficulties, rounded shoulders, potbelly; urinary incontinence; constipation; heartburn; or slow digestion.

26. A method for the treatment of at least one of the following physiological and morphological abnormalities: metabolic disorders such as obesity; water retention; irregular bowel movements; inflammatory diseases such as irritable bowel syndrome, ulcerative colitis, celiac, Crohn's disease); bloating; dyspepsia; gastrointestinal reflux disease; low energy levels; large appetite; dysbiosis; hernia; poor posture related issues such as back pain, hand numbness, spinal dysfunction, joint degeneration, headaches, poor balance, breathing difficulties, rounded shoulders, potbelly; urinary incontinence; constipation; heartburn; or slow digestion; the method including:
measuring the wearer's torso area;
choosing a garment according to claim 1 with the correct fit for the wearer and with the correct compression and/or lift and/or back support required;
instructing the wearer to wear the garment for an initial period of time;
assessing progress after the initial period of time;
assessing whether a garment with greater compression is required for a second period of time.
